(19) United States
(12) Patent Application Publication
(10) Pub. No.: US 2023/0389812 A1
(43) Pub. Date: Dec. 7, 2023

(54) System and Method for In-Ear Detection Using PPG

(71) Applicant: Google LLC, Mountain View, CA (US)

(72) Inventors: Sherk Chung, Mountain View, CA (US); Saket Patkar, Mountain View, CA (US); Ian Atkinson, Mountain View, CA (US)

(73) Assignee: Google LLC, Mountain View, CA (US)

(21) Appl. No.: 18/249,215

(22) PCT Filed: Oct. 19, 2021
(86) PCT No.: PCT/US2021/055621

§ 371 Date: Apr. 14, 2023

Related U.S. Application Data
(60) Provisional application No. 63/094,082, filed on Oct. 20, 2020.

(51) Int. Cl. A61B 5/024; A61B 5/00

(52) U.S. Cl. CPC A61B 5/02416; A61B 5/02438; A61B 5/6815; A61B 5/72; A61B 5/6844; A61B 2562/06; A61B 2562/0257

(57) ABSTRACT

Wearable devices using “legacy” sensors such as proximity sensors or infrared sensors suffer from false positives when, such as when they are placed in a hand, but not inserted on the user's body or on or into the user's ear. Embedding a PPG module or PPG sensor into the wearable device allows for the PPG module to act as a secondary check for the presence of a user wearing the wearable device. In some examples, the PPG module can be used as a sole sensor for the presence or absence of a human user. In other examples, a PPG module can be used to perform a secondary action after the “legacy” sensor performs a first action. For example, the first action can be connecting to a user device while the second action can be opening an application or playing music.

### CROSS-REFERENCE TO RELATED APPLICATIONS

The present application claims the benefit of the filing date of U.S. Provisional Application No. 63/094,082, filed Oct. 20, 2020, entitled System And Method For In-Ear Detection Using PPG, the disclosure of which is hereby incorporated herein by reference.

### BACKGROUND

IR proximity sensors and capacitive touch sensors are typically used for in-ear or on-body detection in wearable devices such as earbuds, headphones, wristbands, and smart watches.

Some earbud devices use proximity sensors to determine whether the device is “on body”, such as for example, whether an earbud is inside the ear of a user. The detection of whether a device is in-ear can be referred to as “in-ear-detection”, or “IED” for short. IED is useful as a means to conserve power and extend battery life, as well as to improve user experience. For example, if a user is playing music through an earbud and takes the earbud out of the ear, detecting that the earbud is no longer in the ear allows the earbud to automatically stop playing music to conserve battery. When the earbud detects that it has been placed back in the ear, the earbud can automatically resume playing the music without any additional user action.

Proximity sensors used in wearable devices generally include an infrared light emitting diode (“LED”) and a photodiode. When the LED is lit, the intensity of infrared light captured by the photodiode can be used to determine the proximity of skin to the sensor. For example, when inside the ear, the ear canal reflects the LED and the photodiode detects a higher intensity oflight from the LED, which translates to the earbud being “in-ear”. When out of the ear, there is no reflection of light from LED back to the photodiode, thus the photodiode captures a low intensity reading which translates to the earbud not being in-ear.

Proximity sensors are used because they are inexpensive and are typically small, at least small enough to be included in wearable devices. However, because the proximity sensor depends on simple light intensity, they are limited in what can be detected. Thus, proximity sensors can sometimes be fooled into generating false positive readings for IED. For example, if the earbud is being held in the hand or in a purse, the proximity sensor can falsely ascertain that the earbud is in the ear, and play music until the battery is drained.

### SUMMARY

Aspects of the disclosed technology improve the performance of IED including improving elimination of false positives, overall accuracy of in-ear or on-body detection, battery performance, and ease of use by users.

Aspects of the disclosed technology include using a photoplethysmography (PPG) sensor for in-ear-detection (IED). Heart rate computation using PPG typically involves emitting light from a green or red LED and taking sensor readings from one or more photodiodes that are placed in contact (or in very close proximity) to the skin. The emitted light penetrates inside the skin, and blood pulsing through the tissue affects the amount of light that is reflected and diffused. The photodiodes measure the light intensity in the tissue and an algorithm translates the variation in intensity to a computed heart rate. Aspects of the disclosed technology include using a digital signal or digital data generated from a PPG sensor or a proximity sensor.

Aspects of the disclosed technology include using or repurposing a PPG sensor to utilize the DC component of the signal for proximity detection and enabling the PPG sensor to serve as a proximity sensor for in-ear detection. The DC component of the PPG sensor is usually discarded, and the AC component is used to estimate heart rate. An aspect of the technology makes use of the DC component to provide a proximity detector. Aspects of the technology also include using the PPG sensor, which can also detect the presence of a pulse, to estimate a heart rate. The heart rate estimate provides another measure that in conjunction with the DC signal or another proximity sensor to verify that the earbud is actually being worn rather than in a pocket. Thus, by making use of the DC component of the PPG signal for proximity detection and the AC component for pulse detection, the PPG sensor can be used as a higher accuracy IED sensor than the typical proximity sensor alone.

Aspects of the disclosed technology include the detection of a heart rate by using a PPG sensor to improve the accuracy of IED by eliminating false positives. Devices that support PPG and are being worn will result in the PPG being able to detect a heart rate. Conversely, if a heart rate cannot be detected, it is likely that the device is not being worn, but could be in a situation that triggers a false positive IED event such as when the device is in a pocket or being held in a hand.

Aspects of the disclosed technology include verifying or checking a signal from a proximity sensor through the use of a PPG sensor. Information derived from the PPG sensor can be used to continue, stop, or cause additional actions to occur on the user device or another device.

Aspects of the disclosed technology include a wearable device, comprising a sensor, the sensor configured to detect a signal related to a physiological parameter associated with a human subject and generate a direct current (DC) signal and an alternating current (AC) signal associated with the detected physiological parameter and a processing device coupled to the sensor and to a memory. The memory can store instructions that cause the processing device to detect the DC signal and the AC signal; determine a heart rate from the AC signal; determine if the wearable device is being worn by the human subject based on detection of the DC signal and the heart rate determination.

Additional aspects of the disclosed technology include a wearable device, comprising a sensor, the sensor configured to detect a signal related to a physiological parameter associated with a human subject and generate a direct current (DC) signal and an alternating current (AC) signal associated with the detected physiological parameter and a processing device coupled to the sensor and to a memory. The memory can store instructions that cause the processing device to detect the DC signal and the AC signal; determine a heart rate from the AC signal; determine if the wearable device is being worn by the human subject based on detection of the DC signal and the heart rate determination. The instructions can cause the processing device to determine the heart rate by detecting a heart rate from the AC signal for a predetermined period of time. The instructions can cause the processing device to authenticate the human subject based on a characteristic of the received DC signal or AC signal. The instructions can cause the processing device to maintain an authentication of the human subject as long as a DC signal or AC signal is received by the device. The instructions can cause the device to remain in an active state upon the processing device determining that the wearable device is being worn by the human subject. The instructions can cause the processing device to determine that the wearable device is not being worn by the human subject based on absence of the heart rate being detected. The instructions can cause the device to enter a low-power state upon the processing device determining that the wearable device is no longer being worn by the human subject. The instructions can cause the processing device to determine proximity of the wearable device to a portion of the human subject's anatomy based on the DC signal detected. The wearable device can be an earbud configured for insertion into the ear of the human subject. The sensor can be a photoplethysmography (PPG) sensor.

Additional aspects of the disclosed technology include a wearable device, comprising a first sensor that detects proximity to a portion of human anatomy based on measurement of at least one electrical property and generates a proximity signal and a second sensor that detects a signal related to a physiological parameter associated with a human subject and generates a direct current (DC) signal and alternating current (AC) signal from the signal related to the detected physiological parameter; and a processing device coupled to the sensor and to a memory. The memory can store instructions that cause the processing device to detect the proximity signal, detect a heart rate from the AC signal, and determine if the wearable device is worn by the human subject based on detection of the proximity signal and detection of a heart rate.

The first sensor can comprise a capacitive electrode and the second sensor can comprise a photoplethysmography (PPG) sensor. The first sensor can comprise a light sensor and the second sensor can comprise a photoplethysmography (PPG) sensor. A determination of whether the wearable device is worn by the human subject can be based upon a weighted combination of the proximity signal and either the DC signal or the AC signal obtained from the PPG sensor. The processing device can determine that the wearable device is worn by the human subject when the weighted average crosses a predetermined threshold. The memory can store a photoplethysmography algorithm, the algorithm generating a confidence metric, and the confidence metric being used to verify that the human subject is wearing the device. The device can be an earbud, a watch, a ring, a pendant, or over-ear headphones. The device can be configured to perform an action upon determination of the wearable device being worn by the human subject. The device can be configured to determine if a human subject is wearing the device based on a first detection and a second detection, the first detection based on a signal from the first sensor and the second detection based on a signal from the second sensor. The second detection can occur after the first action. The device can initiate a first action upon the first detection and continue the first action, or start a second action, upon the second detection. The first action can be playing music. The second detection can be periodically performed. The first action can be discontinued upon the second detection indicating that the human subject is no longer wearing the device. The second detection can be determining if a heart rate is within a range. The first action or second action can be connecting to a second user device, such as a smartphone, laptop, or other device.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1A is a schematic view of a PPG module according to aspects of this disclosure.

FIG. 1B is a schematic drawing of electronics according to aspects of this disclosure.

FIG. 2 is a schematic view of a proximity sensor.

FIG. 3 is a schematic view of a touch sensor.

FIG. 4A is an illustration of a wearable user device according to aspects of this disclosure.

FIG. 4B is an illustration of a wearable user device according to aspects of this disclosure.

FIG. 5 is a schematic diagram of communication within a device according to aspects of this disclosure.

FIG. 6 is a schematic diagram of communication within a device according to aspects of this disclosure.

FIG. 7 is a schematic diagram of communication within a device according to aspects of this disclosure.

FIG. 8 is a flowchart of an example method according to aspects of this disclosure.

FIG. 9 is a flowchart of an example method according to aspects of this disclosure.

FIG. 10 is a flowchart of an example method according to aspects of this disclosure.

FIG. 11 is a flowchart of an example method according to aspects of this disclosure.

FIG. 12 is a diagram illustrating aspects of this disclosure relating to pre-processing events on a user device prior to full PPG confirmation.

### DETAILED DESCRIPTION

Generally, and as non-limiting examples, as used in this disclosure, a “PPG sensor” refers to a photodiode or other sensor which is capable of measuring light. In some examples, the light for the PPG sensor will arrive from an LED or other light source. “PPG data” can generally refer to the readings from a PPG photodiode. A “PPG algorithm” can generally refer to an algorithm that translates or uses PPG data to generate an estimated heart rate. As one non-limiting example, a “PPG system” can generally refer to a combination of a PPG sensor, a CPU or other computing device which can include memory, and a PPG algorithm which can read PPG data and generate an estimated heart rate.

As used in this disclosure, in-ear detection, “IED” or on-body detection can be used interchangeably. A person of skill in the art will recognize these terms to mean the insertion, wearing, or presence upon, or otherwise the use of a user device designed to be used in conjunction with the body of a user.

As used in this disclosure, a touch sensor is intended to describe a device used to determine whether human skin is in contact with the surface of an electrode, typically through capacitance measurements. When the capacitance moves into a range that denotes it is in contact with skin, the device is deemed to be in-ear.

As used in this disclosure, a proximity sensor is intended to describe devices which use an LED and a photodiode to detect proximity. Even though different light sources and light sensors can be used, the combination of an LED plus a photodiode is typically used due to cost and size benefits. In consumer electronics, the LED, photodiode, and related circuitry are often packaged into a single discrete component, further improving cost and size.

IED sensors including proximity and touch sensors are referred to herein as “legacy” IED sensors for convenience. A person of skill in the art should understand that various sensors described herein can contain necessary processing, electronics, and analog to digital converters integrated into the sensor as required.

### OVERVIEW

Wearable devices using “legacy” sensors such as proximity sensors or infrared sensors suffer from false positives such as when they are placed in a hand, but not worn or used as intended, e.g., not inserted into the user's ear. Embedding a PPG module or PPG sensor into the wearable device allows for the PPG module to act as a secondary check for the presence of a user wearing the wearable device. In some examples, the PPG module can be used as a sole sensor for the presence or absence of a human user. In other examples, a PPG module can be used to perform a secondary action after the “legacy” sensor performs a first action. For example, the first action can be connecting to a user device while the second action can be opening an application or playing music.

In some examples, a DC signal or DC data generated from a PPG can be used in conjunction with data or signal generated from a legacy sensor to detect that a device is in-ear. In other examples, a heart beat signal or heart beat data generated from a PPG based on an AC signal or AC data, can be used in conjunction with data or a signal generated from a legacy sensor.

### EXAMPLE SYSTEMS

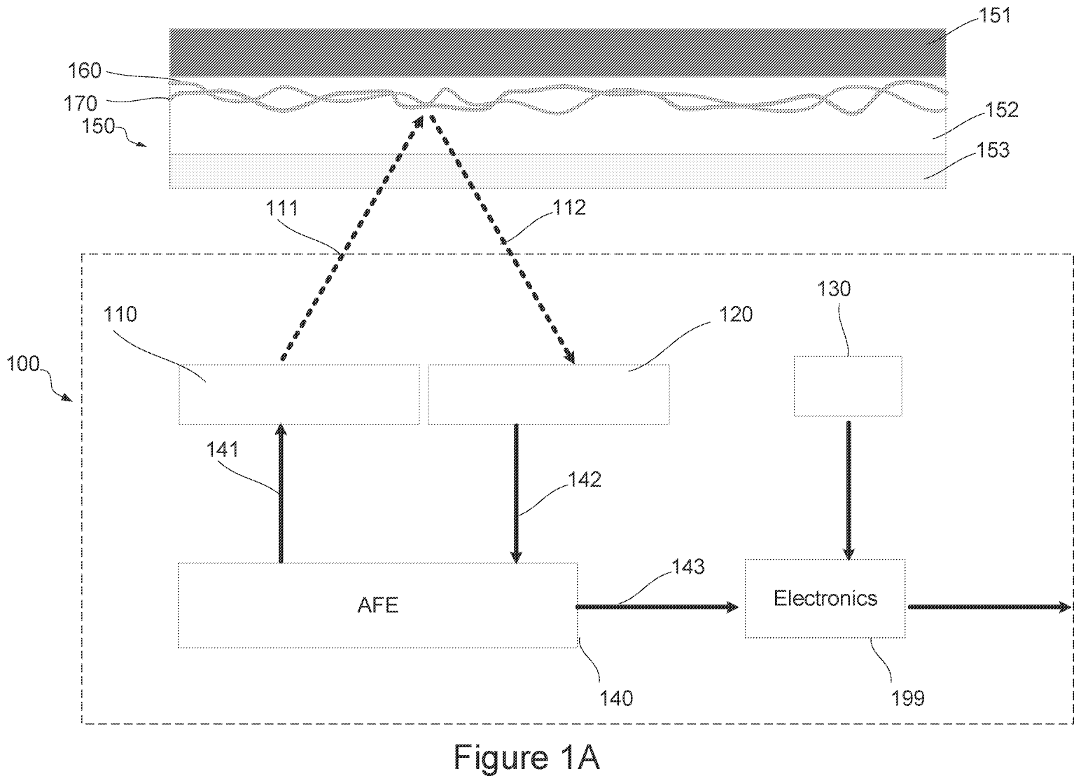

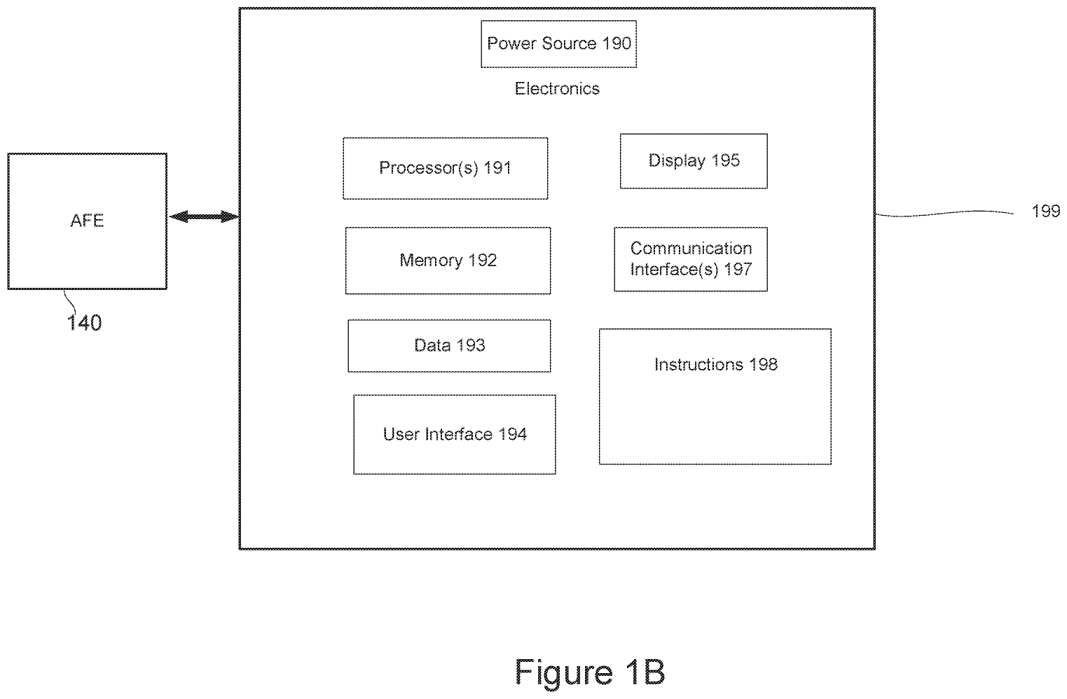

FIG. 1A illustrates a device which can be used to perform PPG, module 100. Module 100 can be contained within or a part of a user device, as explained below. Module 100 can comprise a light source, such as light source 110, one or more light sensors capable of detecting light, such as photodetector 120, accelerometer 130, analog front end (AFE) 140, and electronics 199. In some examples, electronics 199 may be some or all of the features of electronics 199 described below with reference to FIG. 1B. In other examples, features, operations, or components of AFE 140 and electronics 199 may be exchanged or combined in various permutations.

PPG module 100 can generate a direct current signal (DC signal) or an alternating current (AC) signal. The AC signal can be used for heart rate estimation, as explained below. The DC signal however can be used to determine the presence or absence of a human heart beat, and as explained below.

Rays 111 and 112 are light rays, with the arrow indicating the direction in which the light travels. The light can be incident on a dermis, such as skin 150. Although skin 150 is shown, it is possible that the device is applied to other parts of a human body, such as for example, a nail or soft tissue.

FIG. 1A illustrates a light source 110. One example of a light source is a light-emitting diode (LED). An LED is a semiconductor light source which emits light responsive to electricity flowing through it. Electrons in the semiconductor recombine with electron holes, releasing energy in the form of photons. LEDs can be engineered or chosen to emit light at a particular wavelength or range of wavelengths. In other examples, light source 110 can be made of any commercially available source of light, such as lasers, specially designed semiconductors, incandescent light, electrodeless lamps, or halogen lamps. In other examples, light source 110 can further be made of one or more light sources configured to generate light of different wavelengths, such as an LED configured to generate red light which is close to a wavelength of 660 nm, an LED configured to generated green light which is close to a wavelength of 530 nm. These different light sources may be chosen to measure different aspects of a cardiovascular system when performing PPG. For example, green light may provide information regarding a heartbeat while red light may provide information about blood oxygen saturation, due to the relative absorption and reflection of these colors within the cardiovascular system.

A photodetector, such as photodetector 120, can be a semiconductor device that converts light into an electrical current. The photodetector can generate a current which is proportional to the number of photons hitting the surface. As electricity is generated when photons are absorbed in the photodetector, the photodetector can act as a sensor for light. The photodetector can be any device which is capable of sensing intensities and/or wavelengths of light. Photodetector 120 can be a photodiode or a photosensor. In some examples, photodetector 120 can be chosen to be more sensitive to specific wavelengths of light. In some examples, photodetector 120 can be chosen or configured to be more sensitive or only sensitive to green light while another photodetector can be configured to be more sensitive or only sensitive to red light. Photodetector 120 can also be made of an array of photodetectors. Additional circuitry, calibration, or electronics can be incorporated into the photodetectors, AFE 140, or electronics 199 to ensure a better signal to noise ratio and reduce the effect of ambient light.

In some examples, readings from photodetector 120 can be converted to digital samples at AFE 140 which are forwarded to a CPU of electronics 199, where a PPG algorithm uses PPG data 143 to generate a heart rate estimate. Peak detection techniques, which can use either a time domain or a frequency domain algorithm, can be used to estimate heart rate from PPG data, but the presence of motion artifacts (MA) can make accurate peak detection challenging. Motion artifacts can occur when a user is not relatively still, causing motion in a portion of the body to change the reflected light being received by photodetector 120. For example, a MA generated when a user is swinging his or her arm can trick PPG algorithms to lock onto an incorrect peak or mask the true peak associated with the heart rate of the user.

AFE 140 can contain an LED driver and an analog-to-digital converter (ADC). An ADC converts an analog signal into a digital signal. An LED driver can “drive” or control light source 110. AFE 140 can be used to drive light source 110 through a drive signal 141. AFE 140 can also receive an analog signal 142 from photodetector 120. In some examples, AFE 140 can be part of electronics 199, or components of electronics 199, described in more detail below, can be included in AFE 140. AFE 140 can generate from the analog signal received PPG data, and transfer this information to electronics 199, through signal 143. Signal 143 can be digital or analog. In some examples, signal 143 is forwarded to a processor within electronics 199.

Accelerometer 130 can be any electromechanical device which is configured to measure acceleration responsive to acceleration forces. Accelerometer 130 can generate vectors reflecting acceleration in one or more independent dimensions. In order to identify peaks created by MA, PPG modules are typically accompanied by an accelerometer. A person of skill in the art will appreciate how data from an accelerometer can be used in conjunction with data from or derived from photodetector 120 in a PPG algorithm in a time-domain adaptive filter to cancel out noise generated by motion. In some examples, accelerometer data can be used in a Fourier transform to identify MA peaks in the frequency domain. Despite these techniques, the cancellation of a motion artifact is difficult and has significant effects on the accuracy. It is also known in the art that cancellation of MA is difficult, which results in lower-than-desired accuracy of the heart rate estimates when the user is in motion, particularly for wrist-worn devices like medical bands and smartwatches where a user is more likely to move his or her arm and the amount of blood being transmitted through the veins and arteries is subject to a greater amount of change based on this motion. In addition, derivative algorithms which use the information generated from the PPG-based heart rate would inherit the errors introduced by the MAs. For example, other algorithms that depend on PPG-based heart rate can include energy expenditure, breathing rate, atrial fibrillation, sleep analysis, and stress analysis.

Also illustrated in FIG. 1A is skin 150, with a hypodermis layer 151, a dermis layer 152, and an epidermis layer 153 which may contain vein 160 and artery 170. Light generated from light source 110, such as ray 111, can be emitted from module 100 to skin 150. Some of the light emitted from the light source penetrates the skin and is reflected back to photodetector 120, such as ray 112. The reflected light is used to compute an estimated heart rate. Light that reflects off or is transmitted back from these layers is useful for the purpose of PPG.

Variations in the light received by the photodetector can be used to determine various aspects of a cardiovascular system, such as the heart rate, pulse, oxygen saturation in the blood, or other health-related information. In some examples, a wave form can be derived from the continuous or near-continuous monitoring of light received by photodetector 120. Light source 110 and photodetector 120 can be connected with electronics 199 or AFE 140 to control the emission of light, and to monitor and analyze the light received from skin 150.

It is to be understood that although module 100 is illustrated with a specific configuration, other arrangements of these components are within the scope of this disclosure. In other examples, module 100 can be included or arranged within user devices, such as a mechanical watch, a smart watch, a smart ring, a cell phone, earbud, headphone, armband, or a laptop computer. In other examples, module 100 can be integrated into jewelry, such as a pendant, necklace, bangle, earring, armband, ring, anklet, or other jewelry.

FIG. 1B illustrates additional aspects of electronics 199. Although the description in FIG. 1B is given with respect to electronics 199, a person of skill in the art should understand that in some examples AFE 140 and electronics 199 can be combined or operate collectively. Illustrated in FIG. 1B is a bidirectional arrow indicating that communication between AFE 140 and electronics 199 can occur.

Electronics 199 may contain a power source 190, processor(s) 191, memory 192, data 193, a user interface 194, a display 195, communication interface(s) 197, and instructions 498. The power source may be any suitable power source to generate electricity, such as a battery, a chemical cell, a capacitor, a solar panel, or an inductive charger. Processor(s) 191 may be any conventional processors, such as commercially available microprocessors or application-specific integrated circuits (ASICs); memory, which may store information that is accessible by the processors including instructions that may be executed by the processors, and data. Memory 192 may be of a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 193 of electronics 199 may be retrieved, stored or modified by the processors in accordance with the instructions 198. For instance, although the present disclosure is not limited by a particular data structure, data 193 may be stored in computer registers, in a relational database as a table having a plurality of different fields and records, XML documents, or flat files. Data 193 may also be formatted in a computer-readable format such as, but not limited to, binary values, ASCII or Unicode. Moreover, data 193 may comprise information sufficient to identify the relevant information, such as numbers, descriptive text, proprietary codes, pointers, references to data stored in other memories (including other network locations) or information that is used by a function to calculate the relevant data.

Instructions 198 may control various components and functions of PPG module 100. For example, instructions 198 may be executed to selectively activate light source 110 or process information obtained by photodetector 120. In some examples, algorithms can be included as a subset of or otherwise as part of instructions 198 included in electronics 199. Instructions 198 can include algorithms to interpret or process information received, such as information received through or generated by analyzing a ray received at a photodetector, PPG signal 143, or information stored in memory. For example, physical parameters of the user can be extracted or analyzed through algorithms. Without limitation the algorithms could use any or all information about the waveform, such as shape, frequency, or period of a wave, Fourier analysis of the signal, harmonic analysis, pulse width, pulse area, peak to peak interval, pulse interval, intensity or amount of light received by a photodetector, wavelength shift, or derivatives of the signal generated or received by photodetector 120. Other algorithms can be included to calculate absorption of oxygen in oxyhemoglobin and deoxyhemoglobin, heart arrhythmias, heart rate, premature ventricular contractions, missed beats, systolic and diastolic peaks, and large artery stiffness index. In yet other examples, artificial learning or machine learning algorithms can be used in both deterministic and non-deterministic ways to extract information related to a physical condition of a user such as blood pressure and stress levels, from, for example, heart rate variability. PPG can also be used to measure blood pressure by computing the pulse wave velocity between two points on the skin separated by a certain distance. Pulse wave velocity is proportional to blood pressure and that relationship can be used to calculate the blood pressure. In some examples, the algorithms can be modified or use information input by a user into memory of electronics 199 such as the user's weight, height, age, cholesterol, genetic information, body fat percentage, or other physical parameter. In other examples, machine learning algorithms can be used to detect and monitor for known or undetected health conditions, such as an arrhythmia, based on information generated by the photodetectors and/or processors.

User interface 194 may be a screen which allows a user to interact with PPG module 100, such as a touch screen or buttons. Display 195 can be an LCD, LED, mobile phone display, electronic ink, or other display to display information about PPG module 100. User interface 194 can allow for both input from a user and output to a user. In some examples, the user interface 194 can be part of electronics 199 or PPG module 100, while in other examples, the user interface can be considered part of a user device.

Communication interface(s) 197 can include hardware and software to enable communication of data over standards such as Wi-Fi, Bluetooth, infrared, radio-wave, and/or other analog and digital communication standards. Communication interface(s) 197 allow for electronics 199 to be updated and information generated by PPG module 100 to be shared to other devices. In some examples, communication interface(s) 197 can send historical information stored in memory 192 to another user device for display, storage, or further analysis. In other examples, communication interface(s) 197 can send the signal generated by the photodetector to another user device in real-time or afterwards for display on that device. In other examples, communication interface(s) 197 can communicate to another PPG module. Communication interface(s) 197 can include bluetooth, Wi-Fi, Gazelle, ANT, LTE, WCDMA, or other wireless protocols and hardware which enable communication between two devices.

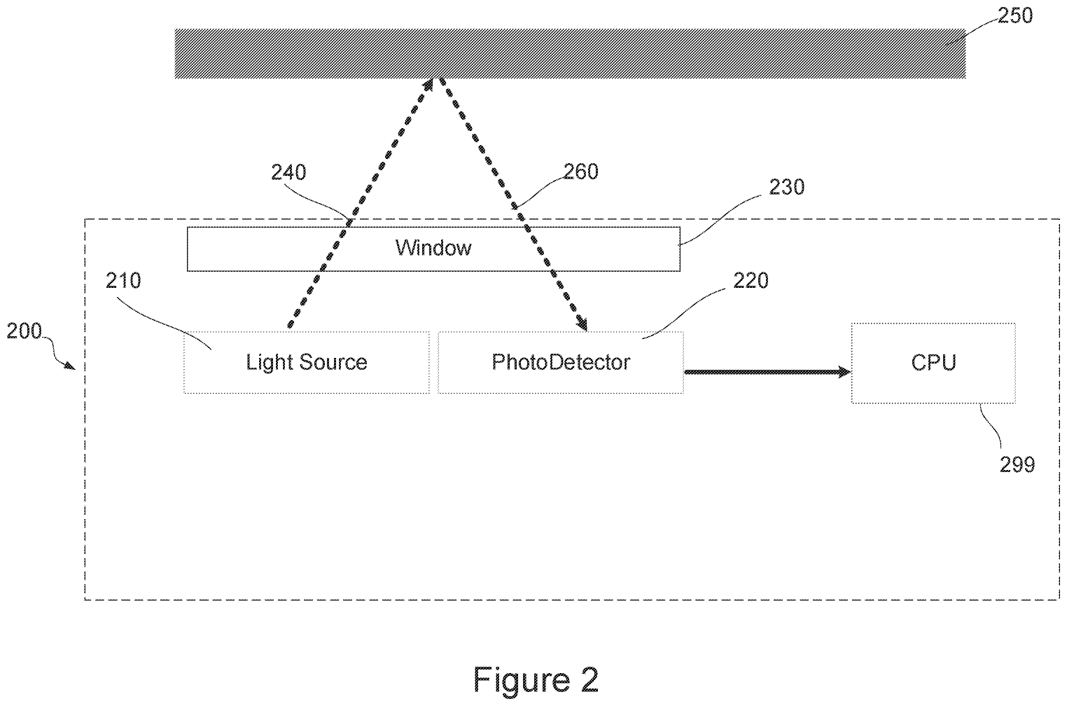

FIG. 2 illustrates a proximity sensor 200. Proximity sensor 200 can be contained within or a part of a user device, as explained below. Proximity sensor 200 can be designed to detect proximity of the proximity sensor to a human user. Proximity sensor 200 can comprise a light source, such as light source 210, one or more light sensors capable of detecting light, such as photodetector 220, both of which can be in communication with and controlled by a processor, such as CPU 299. Light source 210 can be similar to light source 110. Photodetector 220 can be similar to photodetector 120. CPU 299 can be similar to electronics 199 described above. In other examples, CPU 299 can contain any combination of elements described with respect to electronics 199 described above.

Also illustrated in FIG. 2 is a window 230 through which a beam of light, beam 240, which can be generated by light source 210, can be emitted from the proximity sensor and towards human skin, 250. A reflected beam of light, beam 260, can be reflected from the human skin, pass through window 230, and be incident upon and captured by photodetector 220. Photodetector 220 can convert or interpret information from beam 260. This information can be transmitted to CPU 299 from photodetector 220. Window 230 can be made from any material fitted to house the electronics discussed and allow light to transmit through it, such as glass or glass composites.

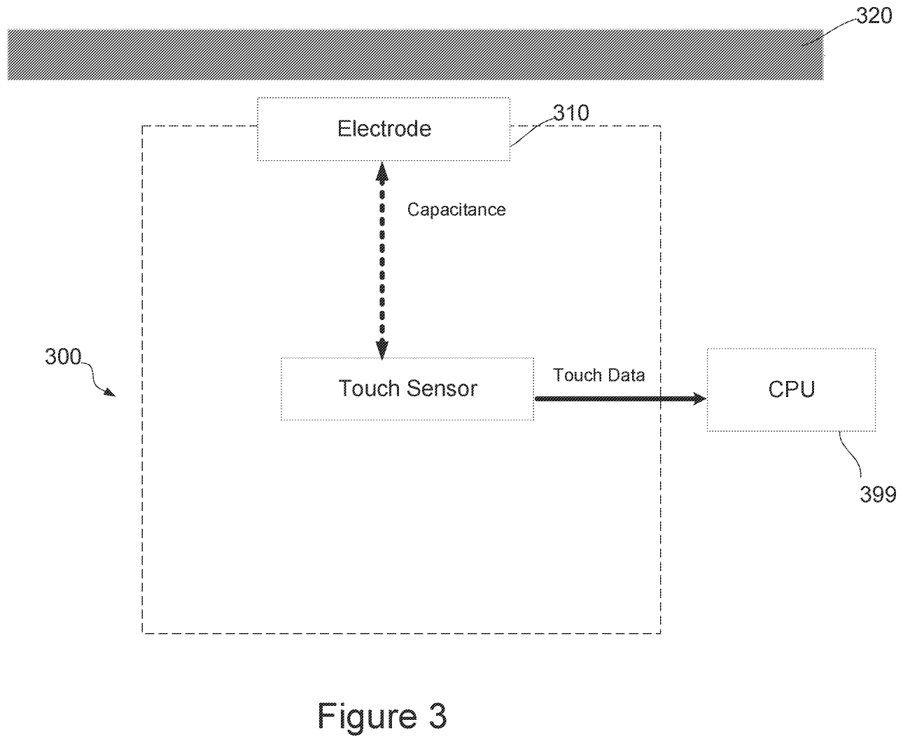

FIG. 3 illustrates a touch sensor 300. Touch sensor 300 can be used to determine whether human skin is in contact with the surface of an electrode, typically through capacitance measurements. When the capacitance moves into a range that denotes it is in contact with the skin, the device is deemed to be in-ear.

As illustrated in FIG. 3, touch sensor 300 can comprise an electrode 310 and a CPU 399. CPU 399 can be similar to electronics 199 described above. In other examples, CPU 299 and CPU 399 can be the same CPU. When electrode 310 is in contact with human skin or another surface, such as surface 320, the capacitance of touch sensor 300 can change. This change in capacitance can indicate the proximity of the touch sensor 300 to a device.

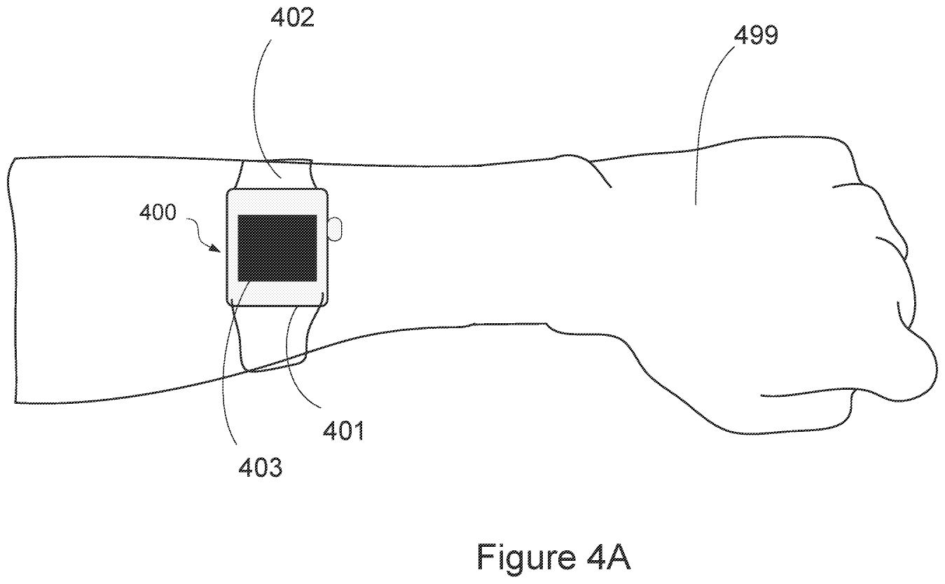

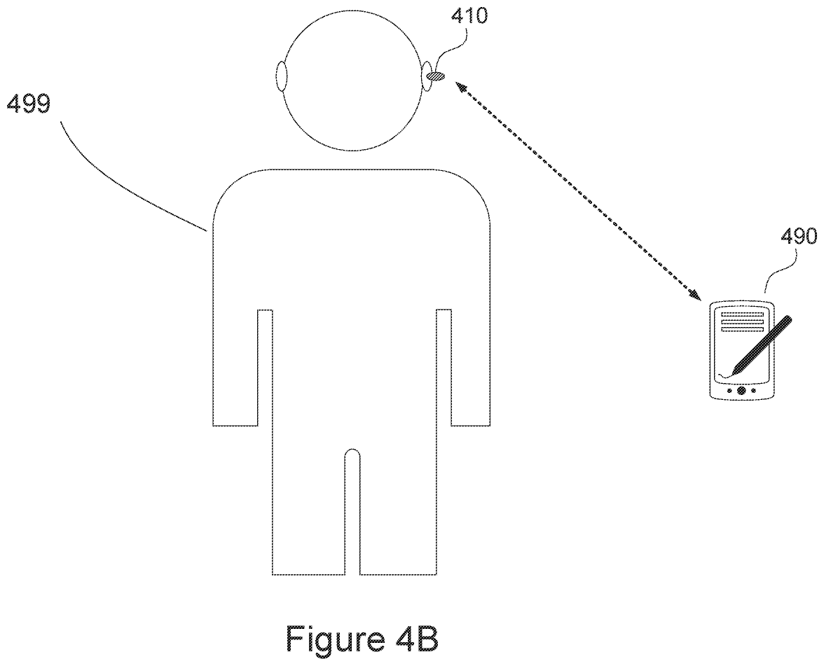

FIG. 4A illustrates a user device, 400, which can be worn by a user, such as user 499. The user device can include a housing 401, and a strap 402. Housing 401 can have components such as a back portion, which will contact the skin of user 499. The back portion can contain a glass portion which will allow light to pass through the back portion. For example, light can be generated from other components contained within housing 401, such as a light source. User device 400 and housing 401 can also have a user interface which allows user 499 to interact and view information from user device 400. The user interface can be part of a touchscreen or other device. Additional components which can be included in user device 400 or in housing 401 are further described above with reference to FIGS. 1A, 1B, 2, and 3. The housing can further be of an appropriate thickness to include the components described in FIGS. 1A and 1B. Strap 402 can be a strap to hold the user device on a user, such as one made from metal, leather, cloth, or other material. User device 400 can contain, among other components, one or more of: (i) PPG module 100 to perform PPG related functions, (ii) proximity detector 200, and (iii) touch sensor 300, and (iv) a CPU.

Although a smartwatch is illustrated as user device 400, a person of skill in the art will appreciate that user device 400 can take on a variety of forms. User device 400 can be a smartwatch, a health sensor, an earbud or earplug, headphone, or other wearable electronics, a ring, a bangle, an anklet, necklace, or other piece of jewelry.

FIG. 4B illustrates a user device 410. User device 410 is configured in the shape of earbuds, which are configured to be inserted into the ears of a user. User device 410 can contain additional logic and communication interfaces, such as those described with respect to electronics 199, to communicate with other devices, such as for example, smartphone 490 or a laptop. Although not illustrated for simplicity, user device 410 can contain, among other components, one or more of: (i) PPG module 100 to perform PPG related functions, (ii) proximity detector 200, and (iii) touch sensor 300, and (iv) a CPU. As explained in this disclosure, by incorporating a PPG sensor, a “true” insertion into the ear of the user can be identified and false positives can be eliminated.

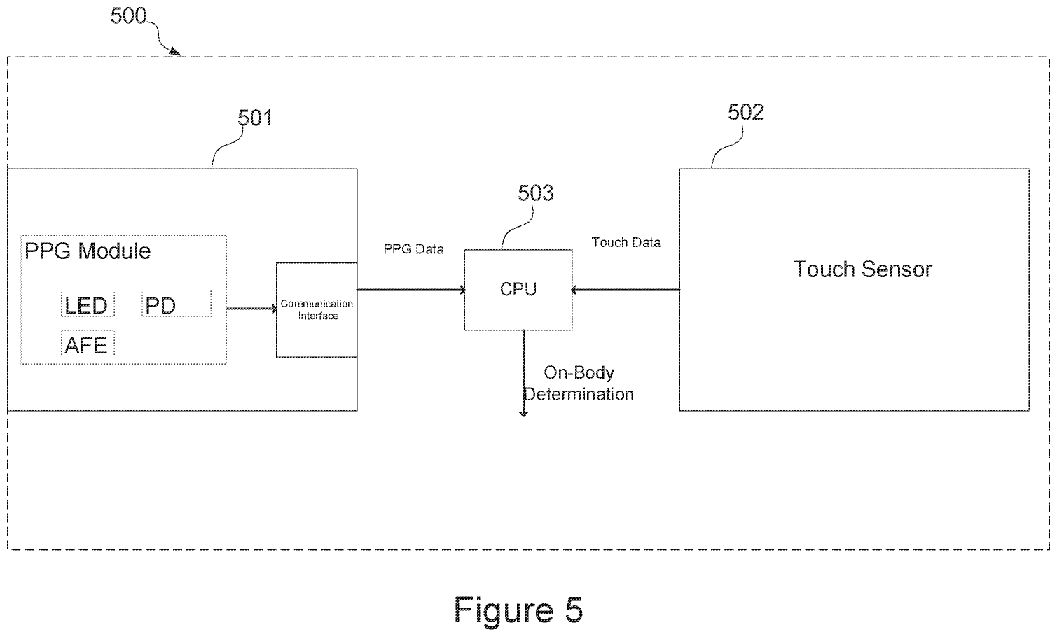

FIG. 5 illustrates a schematic view of an example device 500 incorporating a PPG module and a touch sensor. As illustrated in FIG. 5, device 500 can contain a PPG module 501 sending PPG data to a CPU 503. Device 500 can also contain a touch sensor 502 which can send touch data to CPU 503. PPG module 501 can be similar to PPG module 200; touch sensor 502 can be similar to touch sensor 300; and CPU 503 can be similar to processor 191 or electronics 199. Touch data can include data indicating proximity to a surface. PPG data can include information or an indication that a PPG signal has been detected from the PPG sensor. CPU 503 can receive both the touch data and the PPG data to make determinations of whether or not device 500 is “on body” or “in body” on a user.

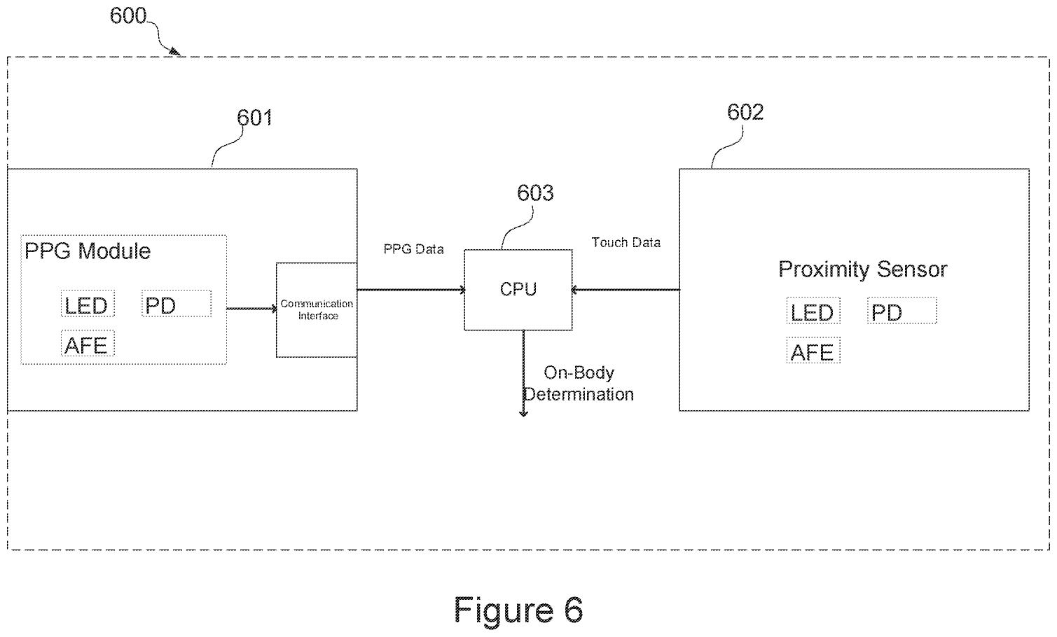

FIG. 6 illustrates a schematic view of an example device 600 incorporating a PPG module and a proximity sensor. As illustrated in FIG. 6, device 600 can contain a PPG module 601 sending PPG data to a CPU 603. Device 600 can also contain a proximity sensor 602 which can send touch data to CPU 603. PPG module 701 can be similar to PPG module 200; touch sensor 502 can be similar to touch sensor 300; and CPU 603 can be similar to processor 191 or electronics 199. Proximity data can include data indicating proximity to a surface. Proximity data can be binary, such as indicating the proximal within a predetermined threshold, or can be coded to indicate the distance to a certain surface. PPG data can include information or an indication that a PPG signal has been detected from the PPG sensor. CPU 603 can receive both the proximity data and the PPG data to make determinations of whether or not device 600 is “on body” or “in-ear” on a user.

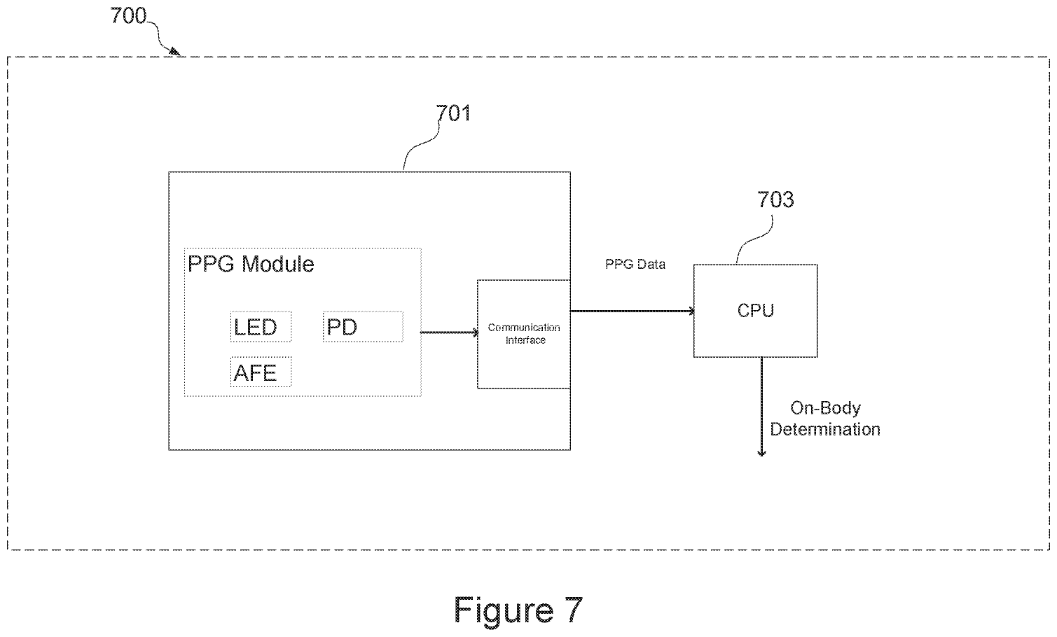

FIG. 7 illustrates a schematic view of an example device 700 incorporating a PPG module. As illustrated in FIG. 7, device 700 can contain a PPG module 701 sending PPG data to a CPU 603. PPG module 701 can be similar to PPG module 200 and CPU 703 can be similar to processor 191 or electronics 199. PPG data can include information or an indication that a PPG signal has been detected from the PPG module. In some examples, the PPG module can detect the heart rate and the devices 700 can be configured to consider that the device is “in-ear” or “on-body” upon detection or calculation of an estimated heart rate. In some examples, when the detected heart rate is within a pre-established range, the estimated or detected heart rate can be considered to be an accurate indication that the device 700 is “in-ear” or “on-body”, as applicable. In some examples, CPU 703 can receive the PPG data to make determinations of whether or not device 700 is “on body” or “in body” on a user.

In some examples, such as those described with reference to FIG. 7, the PPG sensor can completely replace the “legacy” in-ear detection sensor for in-ear detection. In those embodiments where having a single IED sensor is sufficiently accurate, the presence of a PPG sensor means that the legacy IED sensor can be removed for cost and power savings.

In some examples, the PPG sensor of another device can be advantageously used by another user device to perform the methods described in this disclosure. For example, one user device, upon detecting a touch or proximity to a user, can request a PPG signal from another device which belongs to the same user. As one example, one device can be a smart watch, which includes a PPG module, while the other device can be headphones, which over a communication interface, request or receive a PPG signal, to ensure that the headphones are truly “in-ear.”

### EXAMPLE METHODS

As explained below, the following methods can be used to improve the accuracy of in-ear or on-body detection. The following methods can be used to combine a PPG signal with signals from a proximity sensor or touch sensor to improve the accuracy of in-ear detection and reduce the number of false positives as compared to using a proximity sensor or touch sensor alone. In some examples, each sensor or module can determine proximity to a user independently.

In addition, the following methods can be used to replace proximity sensors and touch sensors and allow PPG modules to be used to establish that a device is in-ear or on-body.

In an example, a first PPG system is embedded in an ear-worn device (such as earbuds or headphones), and a second proximity sensor or touch sensor is embedded in the same ear-worn wearable device. Data from the PPG sensor and another sensor can be used as part of determining the device is in-ear or on-body of the user.

In other examples, the PPG module can be in a second device which responds to a request from the ear-worn device to perform a PPG reading. Thus, a wearable device which lacks a PPG module can still utilize the methods described herein.

In yet other examples, the PPG data generated by a user device can be used by a second device to continue authentication of that user on the second device. For example, the second device can have an interface to authenticate a user, such as through a fingerprint reader, PIN, password, login, or other similar mechanism. For example, some smartwatches support near-field communication (NFC) payments, with programs such as Android Pay, and require authentication by the user for payment to be made through that program. Typically, proximity or touch sensors are used on the watch to determine when it is being worn. Once the user is authenticated, the user can be in an authenticated state until the sensors determine that the watch has been removed from the wrist. However, motion of the device can falsely “de-authenticate” the user despite the user having the watch or other wearable device on his or her body.

Thus, in some examples, once authenticated, the second device can request PPG data from a registered user device, and maintain authentication of the user on the second device. For example, the second device can be a smartphone with a financial application which requires a user to be logged into the application, and the second device can maintain the authentication of the user as long as a PPG signal is being received from a registered wearable user device. In some examples, the time period of authentication can be adjusted based on the application so that an intermittent loss of PPG signal or PPG data does not immediately un-authenticate the user. In some examples, the user is kept in an authenticated state until the user is no longer wearing the device. For example, if the wearable device is a smartwatch, the user is kept authenticated until the smartwatch is removed from the wrist as the watch's or other wearable device's on-body detection sensors determine when the device is being worn or not. In some examples, the PPG sensor can be used to authenticate or continue biometric authentication.

In some examples, a PPG signal can be interrupted due to movement of a PPG module generating the PPG signal, such as when a device moves or is loose in-ear or on-body. This can cause a gap regarding the “on-body” status of the PPG signal. In some examples, despite the gap, a biometric comparison of PPG data before and after the loss of a PPG signal or PPG data can be used to determine if the biometrics are similar or an exact match. For example, whenever there is a gap shorter than a threshold in the “on-body status”, instead of de-authenticating the user, a first set of PPG data prior to the gap in data can be compared to a second set of PPG data after the gap. If the two sets of PPG data match, or are within a threshold, the user can still be deemed to be authenticated. If they are not, then, the program using the PPG data can de-authenticate the user. A signature or metric related to the PPG data or PPG signal, which is unique or pseudo-unique to the user can be derived for both the first and second set of PPG data. In some examples, the amount of data samples within each set can depend on the length of the gap.

In other examples, a unique signature related to PPG data can be generated for a particular user and that signature can be used to authenticate the user based on the methods and devices disclosed herein.

In other examples, a legacy IED sensor data can be compared to a threshold, and if the data crosses the threshold for a certain period of time (or a certain number of samples) that sensor deems the device to be in-ear. Similarly, the PPG sensor's data would be compared to the threshold, and if the PPG sensor data crosses the threshold for a certain period of time (or a certain number of samples) the PPG sensor deems the device to be in-ear. The system as a whole would determine that the device is in-ear only if both the legacy IED sensor and the PPG sensor agree that the device is in-ear.

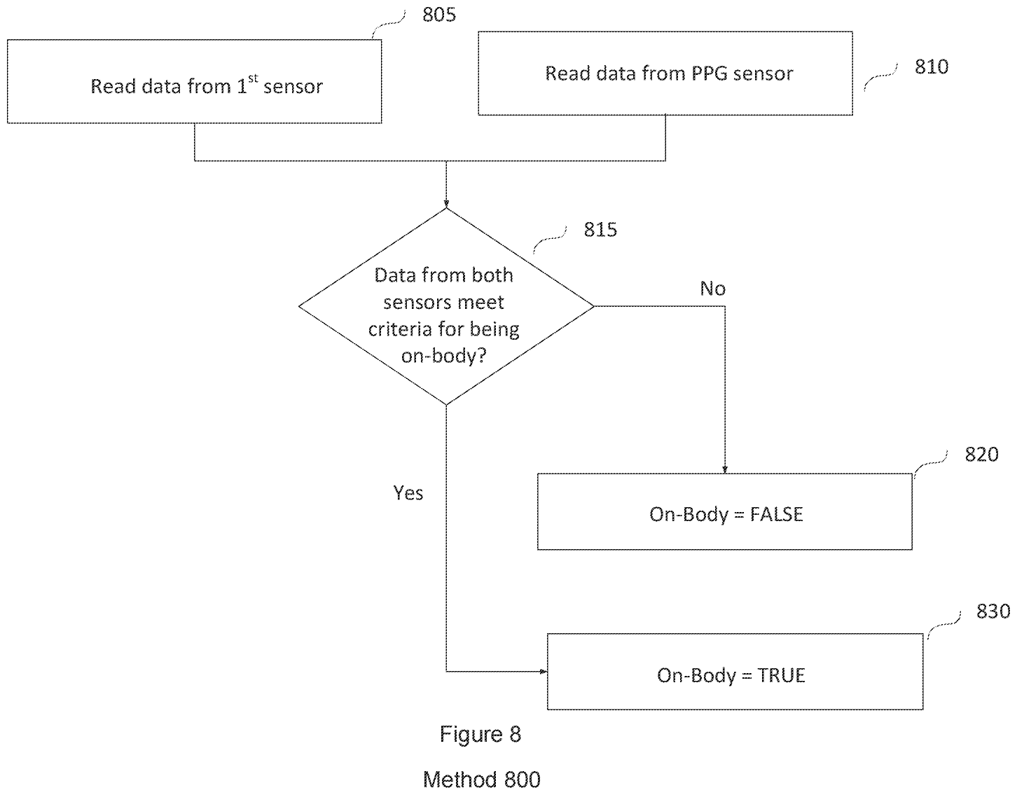

FIG. 8 illustrates an example method 800. Method 800 can be used to combine signals from a first sensor and a PPG sensor to determine if a device containing those sensors is “on-body” or “in-ear”.

At block 805, data can be read from a first sensor. In some examples, the first sensor can be a sensor such as a proximity sensor 200 or touch sensor 300. The first sensor can be a combination of the previously mentioned sensors. In some examples, the first sensor can be configured to generate an alert or a signal when the sensor data crosses a pre-defined threshold.

At block 810, data can be read from a PPG sensor. The PPG sensors can be similar to PPG module 100. In some examples, the presence of a “DC” component or “DC” signal from the PPG sensor can be used as an indication that the device is “in-ear” or “on-body.” In other examples, a combination of a DC signal, AC signal, or other signal from the PPG sensor can be used to determine that the device is “in-ear” or “on-body.”

At block 815, data generated or obtained from the first sensor and the PPG sensor can be analyzed to determine if both sensors meet the criteria for the device being “on-body” or “in-ear.” The method can progress to block 820 or block 825 depending on the result of the analysis at this block. This analysis can be done by a processor or CPU of the device being analyzed.

Method 800 can progress to block 820 if both sensors are not determined to be “on-body” or “in-ear.” At block 820, a determination that the condition of the device being “on-body” or “in-ear” is false.

Method 800 can progress to block 830 if one of the sensors does not meet a criteria for being “on-body” or “in-ear.” At block 830, a determination that the condition of the device being “on-body” or “in-ear” is true.

In some examples, the data from the first sensor and the PPG sensor can be combined prior to calculating a threshold. A weighted average can be computed to determine a metric that is compared to a threshold to determine whether the device is in-ear. An example formula for combining the data is shown below:

IED(n)=W₁(n)·sensor_{legacy}(n)+W₂(n)·sensor_{ppg}(n)

where:
W₁(n) is a weight applied to the 1st sensor data
sensor_{legacy} is the data obtained from the 1st sensor
W₂(n) is a weight applied to the PPG data
sensor_{ppg}(n) is PPG data obtained from the PPG sensor

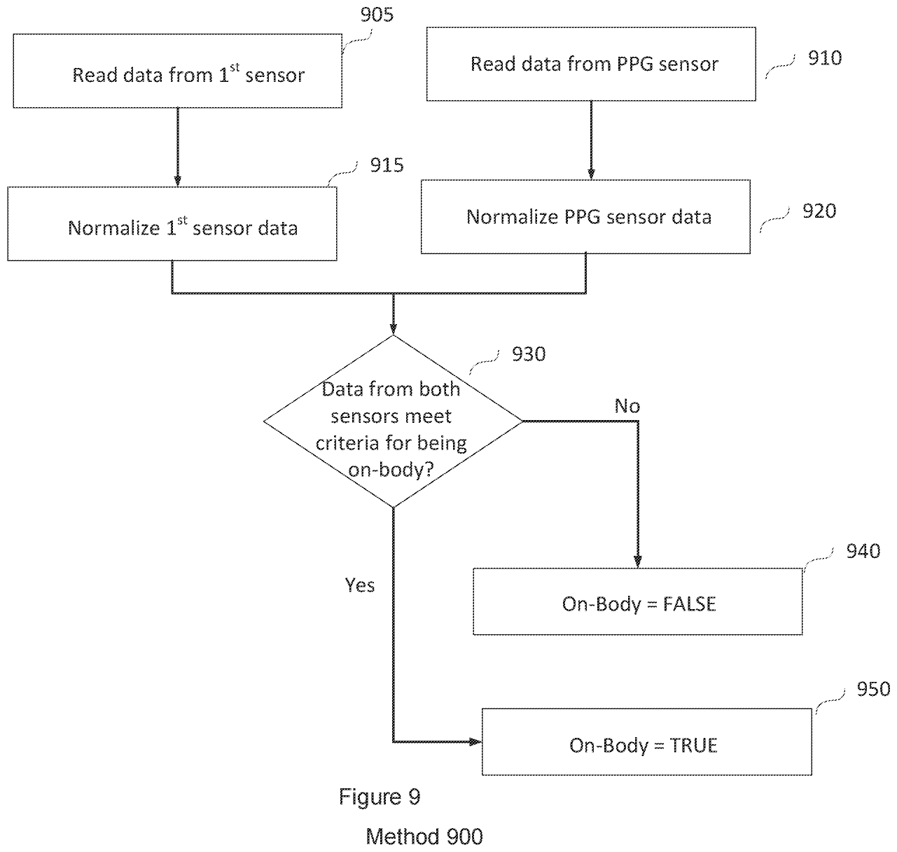

FIG. 9 illustrates an example method 900. Method 900 can be used to combine signals from a first sensor and a PPG sensor to determine if a device containing those sensors is “on-body” or “in-ear”.

At block 905, data can be read from a first sensor. In some examples, the first sensor can be a sensor such as a proximity sensor 200 or touch sensor 300. The first sensor can be a combination of the previously mentioned sensors. In some examples, the first sensor can be configured to generate an alert or a signal when the sensor data crosses a pre-defined threshold.

At block 910, data can be read from a PPG sensor. The PPG sensors can be similar to PPG module 100. In some examples, the presence of a “DC” component or “DC” signal from the PPG sensor can be used as an indication that the device is “in-ear” or “on-body.” In other examples, a combination of a DC signal, AC signal, or other signal from the PPG sensor can be used to determine that the device is “in-ear” or “on-body.”

At block 915, the first sensor data can be pre-processed to generate a normalized value. The pre-processing step can occur at the sensor or at a processor or CPU in data communication with the first sensor. For example, the data can be normalized to range from 0.0 to 1.0. In other embodiments, more sophisticated methods of combining the data can be utilized, such as control loops or kalman filters.

At block 920, the PPG sensor data can be pre-processed to generate a normalized value, similar to the process in block 915.

At block 930 data generated or obtained from the first sensor and the PPG sensor can be analyzed to determine if both sensors meet the criteria for the device being “on-body” or “in-ear.” The method can progress to block 940 or block 950 depending on the result of the analysis at this block. This analysis can be done by a processor or CPU of the device being analyzed. In some examples, the normalized data generated in blocks 915 and 920 can be used for the analysis in block 930.

Method 900 can progress to block 9400 if both sensors are not determined to be “on-body” or “in-ear.” At block 940, a determination that the condition of the device being “on-body” or “in-ear” is false.

Method 900 can progress to block 950 if one of the sensors does not meet a criteria for being “on-body” or “in-ear.” At block 950, a determination that the condition of the device being “on-body” or “in-ear” is true.

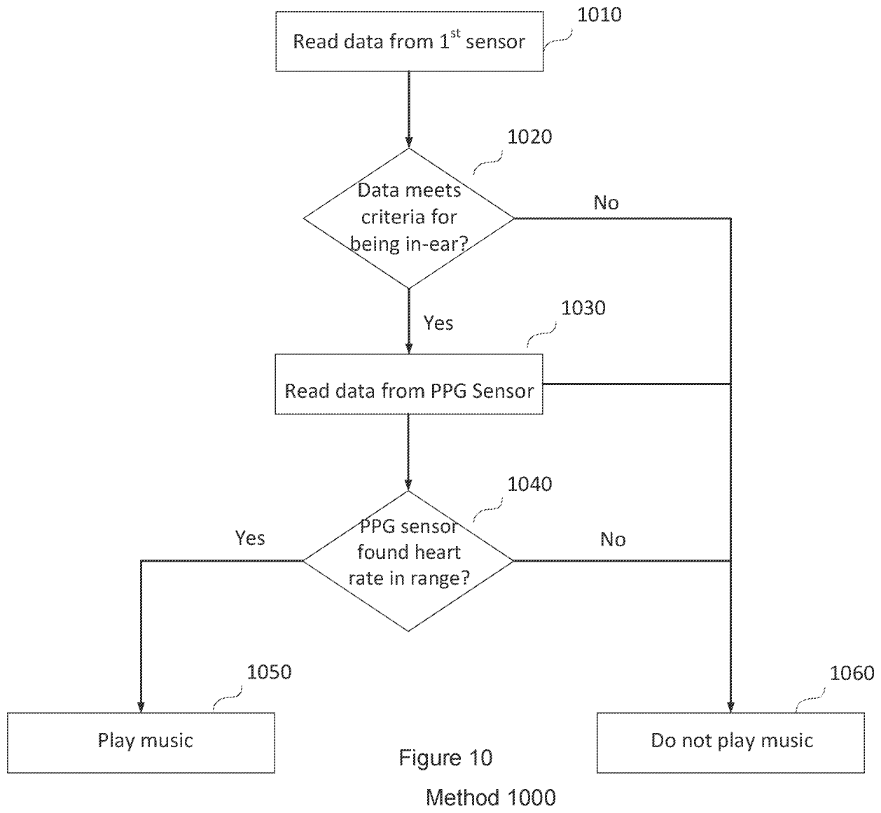

FIG. 10 illustrates aspects of an example method 1000. Method 1000 can be used to automatically perform actions or stop actions from being performed with respect to a user device. For example, in the case of earbuds, or other listening devices, the method can be used to play or stop music. Although reference is given to the specific action of playing music, a person of skill in the art will understand that other actions can be taken. For example, a notification can be sent from the user device to a smartphone or other device connected to the user device. In other cases, a specific application can be launched based on successful detection of the earbud or other device being in-ear or on-body.

At block 1010, data can be read from a first sensor. In some examples, the first sensor can be a sensor such as a proximity sensor 200 or touch sensor 300.

At block 1020, the data obtained in block 1010 can be analyzed for meeting the criteria of being in-ear or not. The method can progress to block 1030 or block 1060 depending on the result of the analysis.

At block 1030, data can be read from a PPG sensor or PPG module. The PPG sensors can be similar to PPG module 100. In some examples, the presence of a “DC” component or “DC” signal from the PPG sensor can be used as an indication that the device is “in-ear” or “on-body.” In other examples, a combination of a DC signal, AC signal, or other signal from the PPG sensor can be used to determine that the device is “in-ear” or “on-body.”

At block 1040, a confidence metric produced by the PPG algorithm can be used in the secondary verification of in-ear detection detected in block 1030. At block 1040 a confidence metric produced by the PPG algorithm is used in the secondary verification of in-ear detection. A confidence metric is a measure of how confident the algorithm is of the accuracy of the estimated heart rate. In some examples, once the first in-ear detection sensor detects the device is in-ear, the PPG sensor is used to compute a heart rate. If the confidence level of the estimated PPG heart rate crosses a threshold, the device is confirmed to be in-ear.

In some examples, two checks can be performed. A first check to determine if the heart rate is within a range, and a second check to determine if the confidence level of the estimated heart rate is sufficiently high to conclude that the heart rate is accurate and thus the device is in-ear.

At block 1050, an action can be taken responsive to the heart rate being found within a predetermined range or confidence interval.

At block 1060, an action can be stopped, reversed, or prevented from occurring based on either sensor not meeting a criteria or range, as described with respect to the steps above.

In some examples, the device can be deemed to be in-ear only if both the primary IED sensor triggers and IED and the PPG algorithm's heart rate output confirms the device is in-ear. In some examples, as the PPG algorithm may take many seconds to compute a heart rate of sufficient accuracy for reliable IED, requiring both to be “true” would mean that IED could take many seconds, which is undesirable in some cases. For example, if an earbud is programmed to automatically start playing music when the device is placed in the ear, it would be a better user experience to start playing music right away, instead of waiting 10 seconds or so to start playing music.

In some examples, the period between detection in the first sensor and the second sensor can be used for two-stage detection. A first detection can be deemed sufficient for the device to start operating as if the device is in-ear. At a later time, a second detection can confirm the detection and allow the operation of the device to continue or reverse the state if the second detection by a second sensor does not confirm the state. For example, when an earbud is placed inside a pocket, a proximity sensor acting as the first stage detection mechanism could denote that the device is in-ear and result in music to begin playing. After 10 seconds, the PPG sensor would determine the device is actually not in the ear, and would turn off the music. Such a method allows for low-latency response to the first stage IED detection to improve user experience, while enabling power savings by turning off the music if the device is not confirmed to be in the ear by the PPG sensor.

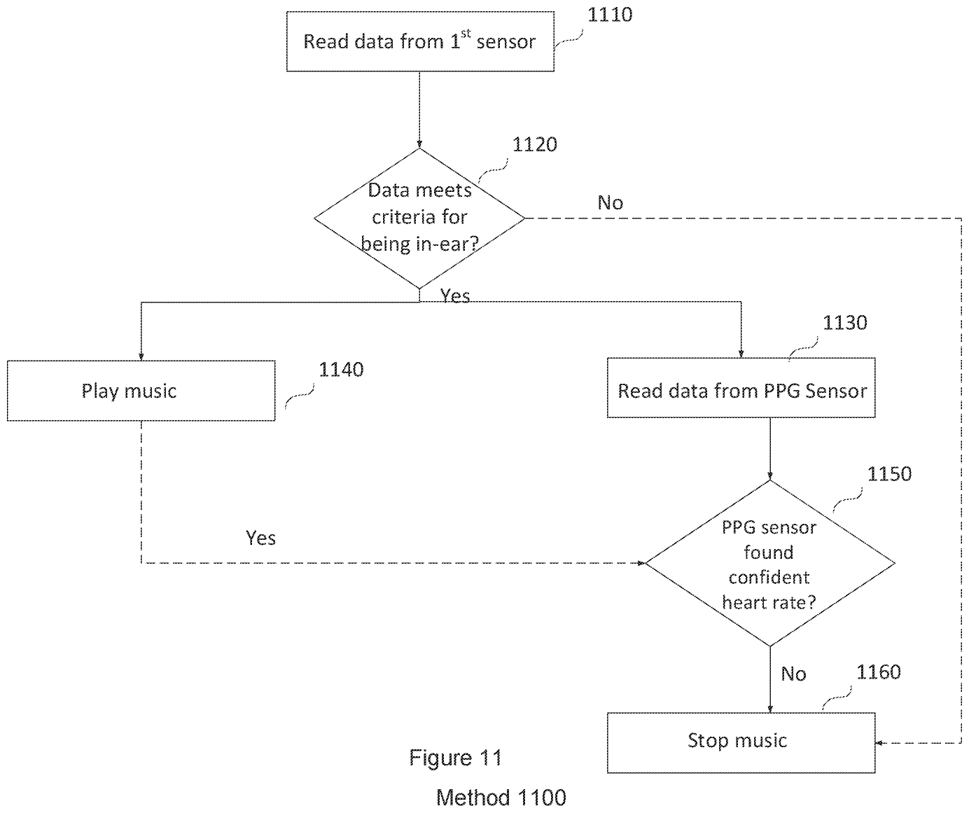

FIG. 11 illustrates aspects of an example method 1100. Method 1100 can be used to automatically perform actions or stop actions from being performed with respect to a user device. For example, in the case of earbuds, or other listening devices, the method can be used to play or stop music. Method 1100 can be used to play music (or another event or activity) prior to a data from a PPG sensor being obtained for faster processing or “pre-processing.” In this manner, the activity can be initiated and then verified for a smaller delay for a user.

At block 1110, data can be read from a first sensor. In some examples, the first sensor can be a sensor such as a proximity sensor 200 or touch sensor 300. The first sensor can be a combination of the previously mentioned.

At block 1120, the data can be analyzed to meet criteria for being “in-ear.” This can include the presence of a DC or an AC signal, or other detection, such as from a proximity sensor.

At block 1130, additional data can be read from a PPG sensor. This can include a DC or an AC signal from a PPG signal or generated at a PPG sensor.

At block 1140, music, or another event can be initiated. In some examples, block 1140 can occur upon certain criteria, such as those specified in block 1120, being met.

At block 1150, additional data can be read from the PPG sensor. The PPG sensor data or related data can be evaluated to determine if a heart rate is detected within a certain confidence range.

At block 1160, music or other events can be stopped upon the criteria specified in block 1150 or block 1120 not being met.

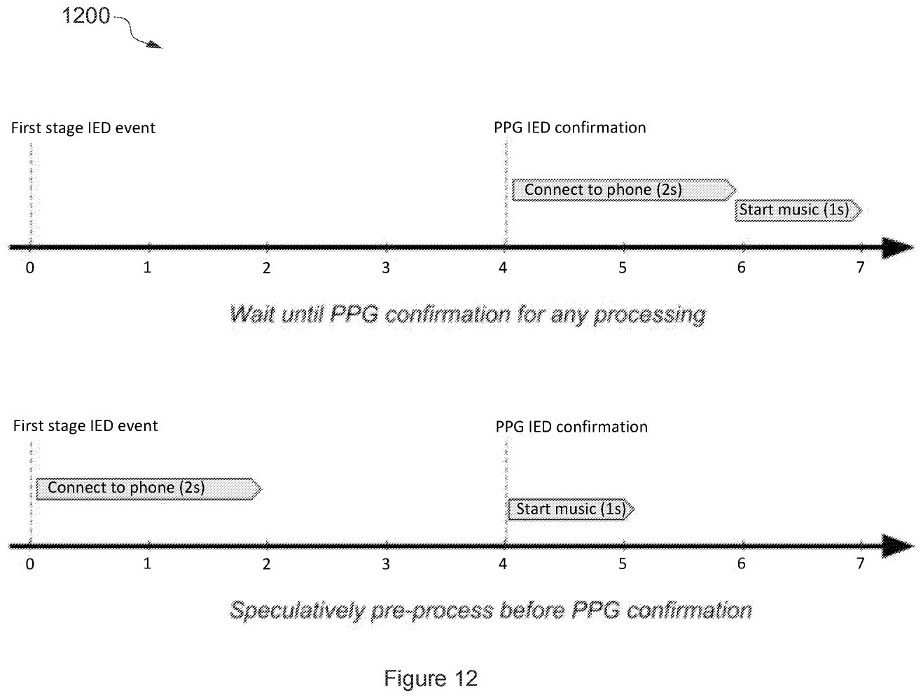

FIG. 12 is a diagram illustrating aspects of this disclosure relating to pre-processing events on a user device prior to full PPG confirmation. As can be seen from FIG. 12, a PPG IED confirmation can be awaited prior to connecting to a user device and starting music. However, in other examples, upon the detection of a first stage IED, certain actions can be taken, such as connecting to a user device, and confirmation of the PPG IED can trigger a second action, such as playing music from the connected user device.

In some examples, the device takes a more conservative approach upon receiving an IED event from the first stage sensor. Instead of assuming the device is in-ear, the device performs some preparatory activities so that if the PPG sensor confirms it is in-ear, it takes less time for earbuds or other device to execute its programmed response. For example, assume the earbud is programmed to play music when it goes in-ear. Upon the first stage IED event, the earbud establishes a connection to the paired smartphone. Upon the second stage PPG confirmation, the earbud then asks the smartphone to start playing music. Using this method allows, for example, a partial response upon first stage IED by establishing a connection to the phone so that it doesn't need to perform that after the 2nd stage PPG confirmation, therefore overall reducing the latency between the time the earbud is placed in ear, and when it starts to play music.

In some examples, the PPG check can occur at pre-determined intervals, which can be defined by the wearable device containing the PPG sensor, or by a connected device or application on the connected device, such as a music application on a smartphone. An action can continue upon the PPG check being passed while an action, such as playing music, can be terminated upon the PPG check failing.

In addition, although examples have been given with reference to HR estimates and metrics derived from HR estimates, a person of skill in the art should recognize that the same techniques can be applied to other metrics derivable from the PPG module, such as blood oxygenation saturation (SPO2).

While this disclosure contains many specific implementation details, these should not be construed as limitations on the scope of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Non-limiting aspects of the disclosed technology can include the following features:

¶1. A wearable device, comprising:
a sensor, the sensor configured to detect a signal related to a physiological parameter associated with a human subject and generate a direct current (DC) signal and an alternating current (AC) signal associated with the detected physiological parameter; and
a processing device coupled to the sensor and to a memory, the memory storing instructions that cause the processing device to:
detect a first signal related to the DC signal;
detect a second signal related to the AC signal;
determine a heart rate based on the second signal;
determine if the wearable device is being worn by the human subject based on detection of the first signal and the heart rate determination.
   ¶2. The device of ¶1, wherein the instructions cause the processing device to determine the heart rate by detecting a heart beat from the second signal for a predetermined time period.
   ¶3. The device of ¶¶1-2 wherein the instructions cause the processing device to authenticate the human subject based on a characteristic of the received first signal or second signal.
   ¶4. The device of ¶¶1-3 wherein the instructions cause the processing device to maintain an authentication of the human subject as long as a first signal or second signal is received by the device.
   ¶5. The device of ¶¶1-4 wherein instructions cause the device to remain in an active state upon the processing device determining that the wearable device is being worn by the human subject.
   ¶6. The device of ¶¶1-5, wherein the instructions cause the processing device to determine that the wearable device is not being worn by the human subject based on absence of the heart rate being detected.
   ¶7. The device of ¶¶1-6 wherein the instructions cause the device to enter a low-power state upon the processing device determining that the wearable device is no longer being worn by the human subject.
   ¶8. The device of ¶¶1-6, wherein the instructions cause the processing device to determine proximity of the wearable device to a portion of the human subject's anatomy based on the first signal detected.
   ¶9. The device of ¶¶1-6, wherein the wearable device is an earbud configured for insertion into the ear of the human subject.
   ¶10. The device of ¶¶1-9, wherein the sensor is a photoplethysmography (PPG) sensor.
   ¶11. A wearable device, comprising:
a first sensor that detects proximity to a portion of human anatomy based on measurement of at least one electrical property and generates proximity data from a proximity signal;
a second sensor that detects a signal related to a physiological parameter associated with a human subject and first data associated with a alternating current (AC) signal from the signal related to the detected physiological parameter; and
a processing device coupled to the sensor and to a memory, the memory storing instructions that cause the processing device to:
   detect the proximity data;
   detect a heart rate based on the first data;
   determine if the wearable device is worn by the human subject based on detection of the proximity data and detection of a heart rate.
      ¶12. The device of ¶11, wherein the first sensor comprises a capacitive electrode and the second sensor comprises a photoplethysmography (PPG) sensor.
      ¶13. The device of ¶¶11-12, wherein the first sensor comprises a light sensor and the second sensor comprises a photoplethysmography (PPG) sensor.
      ¶14. The device of ¶¶11-13 wherein the determination of whether the wearable device is worn by the human subject is based upon a weighted combination of the proximity data and either the first data or second data based upon a direct current (DC) signal generated from the second sensor.
      ¶15. The device of ¶¶11-14 wherein the processing device determines that the wearable device is worn by the human subject when the weighted average crosses a predetermined threshold.
      ¶16. The device of ¶¶11-15 wherein the memory stores a photoplethysmography algorithm, the algorithm generating a confidence metric, the confidence metric being used to verify that the human subject is wearing the device.
      ¶17. The device of ¶11-16 wherein the device is an earbud.
      ¶18. The device of ¶¶11-16 wherein the device is over-ear headphones.
      ¶19. The device of ¶¶11-16 wherein the device is configured to perform an action upon determination of the wearable device being worn by the human subject.
      ¶20. The device of ¶19 wherein the device is configured to determine that the human subject wearing the device based on a first detection and a second detection, the first detection based on data from the first sensor and the second detection based on data from the second sensor.
      ¶21. The device of ¶20 wherein the second detection occurs after the first action.
      ¶22. The device of ¶20 wherein the device initiates a first action upon the first detection and continuing the first action upon the second detection.
      ¶23. The device of ¶22 wherein the first action is playing music.
      ¶24. The device of ¶¶11-20 wherein the second detection is periodically performed.
      ¶25. The device of ¶¶11-20 wherein the first action is discontinued upon the second detection indicating that the human subject is no longer wearing the device.
      ¶26. The device of ¶¶11-20 wherein the second detection is determining if a heart rate is within a range.
      ¶27. The device of ¶¶11-22 wherein the first action is connecting to a second user device.

1. A wearable device, comprising:
a sensor, the sensor configured to detect a signal related to a physiological parameter associated with a human subject and generate a direct current (DC) signal and an alternating current (AC) signal associated with the detected physiological parameter; and
a processing device coupled to the sensor and to a memory, the memory storing instructions that cause the processing device to:
receive a first signal related to the DC signal;
receive a second signal related to the AC signal;
determine a heart rate based on the second signal; and
determine if the wearable device is being worn by the human subject
based on detection of the first signal and the heart rate determination.

2. The device of claim 1, wherein the instructions further cause the processing device to determine the heart rate based on a heartbeat over a predetermined time period from the second signal.

3. The device of claim 1, wherein the instructions further cause the processing device to authenticate the human subject based on a characteristic of the received first signal or second signal.

4. The device of claim 3, wherein the instructions further cause the processing device to maintain the authentication of the human subject as long as the first signal or second signal is being received by the device.

5. The device of claim 1, wherein the instructions further cause the processing device to keep the wearable device in an active state upon the processing device determining that the wearable device is being worn by the human subject.

6. The device of claim 1, wherein the instructions further cause the processing device to determine that the wearable device is not being worn by the human subject based on absence of the heart rate being detected.

7. The device of claim 6, wherein the instructions further cause the processing device to place the wearable device in a low-power state upon the processing device determining that the wearable device is no longer being worn by the human subject.

8. The device of claim 1, wherein the instructions further cause the processing device to determine a proximity of the wearable device to a portion of the human subject's anatomy based on the first signal.

9. The device of claim 1, wherein the wearable device is an earbud configured for insertion into an ear of the human subject.

10. The device of claim 1, wherein the sensor is a photoplethysmography (PPG) sensor.

11. A wearable device, comprising:
a first sensor, the first sensor configured to detect proximity to a portion of human anatomy based on measurement of at least one electrical property, the measurement represented by a proximity signal, and generate proximity data based on the proximity signal;
a second sensor, the second sensor configured to detect a signal related to a physiological parameter associated with a human subject and generate first data associated with an alternating current (AC) signal from the signal related to the detected physiological parameter; and
a processing device coupled to the first and second sensor and to a memory, the memory storing instructions that cause the processing device to:
receive the proximity data;
determine a heart rate based on the first data; and
determine if the wearable device is being worn by the human subject based on the proximity data and the heart rate.

12. The device of claim 11, wherein the first sensor comprises a capacitive electrode and the second sensor comprises a photoplethysmography (PPG) sensor.

13. The device of claim 11, wherein the first sensor comprises a light sensor and the second sensor comprises a photoplethysmography (PPG) sensor.

14. The device of claim 11, wherein the determination of whether the wearable device is worn by the human subject is further based on a weighted combination of the proximity data and either the first data or a second data generated by the second sensor, the second data associated with a direct current (DC) signal from the signal related to the detected physiological parameter.

15. The device of claim 14, wherein the instructions further cause the processing device to determine that the wearable device is worn by the human subject when the weighted average crosses a threshold.

16. The device of claim 11, wherein the instructions comprise a photoplethysmography algorithm, the algorithm generating a confidence metric, and the determination that the wearable device is being worn by the human subject is further based on the confidence metric.

17. The device of claim 11 wherein the device is an earbud.

18. The device of claim 11 wherein the device is over-ear headphones.

19. The device of claim 11 wherein the device is configured to perform an action upon determination of the wearable device being worn by the human subject.

20. The device of claim 11, wherein the determination that the human subject is wearing the device is further based on a first detection and a second detection, the first detection based on data from the first sensor and the second detection based on data from the second sensor.

21. The device of claim 20, wherein the second detection occurs after the first detection.

22. The device of claim 20, wherein the device initiates a first action upon the first detection and continues the first action upon the second detection.

23. The device of claim 22 wherein the first action is playing music.

24. The device of claim 20 wherein the second detection is periodically performed.

25. The device of claim 22, wherein the first action is discontinued upon the second detection indicating that the human subject is no longer wearing the device.

26. The device of claim 20 wherein the second detection is determining if a heart rate is within a range.

27. The device of claim 22 wherein the first action is connecting to a second user device.
